SEQUENCE LISTING (4 sequences)

SEQ ID NO: 1 - DNA, 9481 bp, synthetic construct
  source 1..9481: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgactagtcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcccgtttagtgaaccgtcagatcgcctggagacgccatccacgctgttttgacctccatagaagattctagagctagcgaattcatgacgtcatcacatgccatgaacatcacccccctggctcagctcgccctgctcttctccacgctactgcttccgggaactcaggccgtcgacctggacatcgtgctgagccagagccccgccatcatgagcgcctcccccggagagaaggtgaccatctcctgctccgcctccagctccgtgagctatatgtactggtatcagcagaaacccggaagcagtcccaaaccctggatctataggaccagcaatctggccagcggggtgcccgccagattctccggaagtggcagcggcacatcatacagcctgactatcagcagcatggaagccgaagacgccgccacctactactgtcagcagtaccactcttacccccccaccttcggagccggcaccaaactggagctgaaaagctccggcggcggcggcagcggaggaggaggaggcggatctagcagaagcagcctggaggtgaaactggtggagagcggccccgagctgaagaagccaggagagacagtgaagatcagctgtaaggctagcggatacacattcaccgactacagtatgcactgggtgaaccaggcccccggcaaaggcctgaagtggatggggtggatcaacaccgagacaggcgagccctcctacgccgacgacttcaaaggcagattcgccttcagcctggagaccagcgctagcaccgcctacctgcagatcaacaacctgaagaatgaggacacagccacatacttctgcgccaccgactacggcgattacttcgattactggggacagggaaccaccctgaccgtgagcagcggtaccgagcagaagctgatcagcgaggaagacctgactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatcagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaatgagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagacgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaatcaacctctggattacaaaatttgtgaaagattgactggtattcttaactatgttgctccttttacgctatgtggatacgctgctttaatgcctttgtatcagttaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 2 - protein, 7 aa, synthetic construct
KKKPRVK

SEQ ID NO: 3 - protein, 10 aa, synthetic construct
GRKKRRQRRR

SEQ ID NO: 4 - protein, 18 aa, synthetic construct
KGRKKRRQRRRKKKPRVK

(19) United States
(12) Patent Application Publication
(10) Pub. No.: US 2025/0339535 A1
(43) Pub. Date: Nov. 6, 2025

(54) IN VIVO AND IN VITRO EDITING PREPARATION METHOD FOR CAR-MF TARGETING TUMOR STEM CELLS, AND USE THEREOF

(71) Applicant: SHANDONG UNIVERSITY, Jinan (CN)

(72) Inventors: Xinyi JIANG, Jinan (CN); Chen CHEN, Jinan (CN); Weiqiang JING, Jinan (CN)

(21) Appl. No.: 19/099,808

(22) PCT Filed: Apr. 25, 2023
(86) PCT No.: PCT/CN2023/090490

§ 371 Date: Jan. 30, 2025

(30) Foreign Application Priority Data
Aug. 3, 2022 (CN) 202210925973.0
Aug. 4, 2022 (CN) 202210933047.8

(51) Int. Cl. A61K 40/42; A61K 40/17; A61K 40/31; A61P 35/00; C07K 14/725; C07K 16/28

(52) U.S. Cl. CPC A61K 40/4224; A61K 40/17; A61K 40/31; A61P 35/00; C07K 14/7051; C07K 16/2896; C07K 2317/622; C07K 2319/09

(57) ABSTRACT

An in vivo and in vitro editing preparation method for CAR-MΦ targeting tumor stem cells, and a use thereof. The invention provides a chimeric antigen receptor-macrophage (CAR-MΦ) and a nano-carrier based on self-assembled nano-micelles, which can be applied to immunotherapy of glioma. According to the present invention, (PA)2 peptide nano-micelles loaded with CD133-CAR plasmids are constructed, and a citraconic anhydride-modified dextran, which is a group targeting macrophage specific target CD206, is adopted for modification. The carrier is used to realize CAR editing of macrophage in vivo and in vitro, so as to facilitate the re-education of tumor-related macrophage from an M2 phenotype to an M1 phenotype. At the same time, the surface markers of tumor stem cells are targeted to accurately target the tumor stem cells, phagocytize tumor cells, activate tumor immunity, remodel the tumor inhibition microenvironment and specifically kill brain glioma stem cells, thereby efficiently treating brain glioma.

### REFERENCE TO SEQUENCE LISTING

The instant application contains a Sequence Listing in XML format as a file named “PCT2023027US Sequence Listing.xml”, created on Jan. 27, 2025, of 13,954 bytes in size, and which is hereby incorporated by reference in its entirety.

### CROSS-REFERENCE TO RELATED APPLICATIONS

The present invention claims priority to Chinese Patent Application No. 202210933047.8, filed on Aug. 4, 2022, with the title “In Vivo and In Vitro Editing Preparation Method for CAR-MΦ Targeting Tumor Stem Cells, and Use Thereof” at the China National Intellectual Property Administration. The present invention also claims priority to Chinese Patent Application No. 202210925973.0, filed on Aug. 3, 2022, with the title “In Vivo and In Vitro Editing Preparation Method for CAR-MΦ Targeting Tumor Stem Cells, and Use Thereof” at the China National Intellectual Property Administration. The entire contents of these applications are incorporated herein by reference.

### TECHNICAL FIELD

The present invention belongs to the field of tumor immunotherapy technology, specifically relating to a chimeric antigen receptor, macrophages modified with the chimeric antigen receptor, a nano-carrier based on amphiphilic polymers for targeted delivery, and their applications in the field of glioma immunotherapy.

### BACKGROUND

The information disclosed in this background section is provided solely to enhance the overall understanding of the present invention and should not necessarily be considered as prior art known to those skilled in the art.

Glioma is currently one of the most common and most invasive malignant tumors. Current conventional clinical glioma treatment adopts surgical resection followed by concurrent radiotherapy and chemotherapy. Due to its unclear boundaries, difficulty in complete surgical removal, and the presence of glioma stem cells (GSCs), the prognosis is extremely poor, with less than 10% five-year survival rate after diagnosis and a median survival of 14-16 months.

Some GSC markers are highly expressed on the cell surface. Through modification of specific cells or drug modifications, targeted binding with tumor stem cells can be achieved, accomplishing the goal of targeted therapy. Therefore, GSCs surface markers can serve as targets for tumor immunotherapy. CD133, as a molecule specifically highly expressed in tumor stem cells, is demonstrated in gliomas, colon cancer, and other tumors, and has been confirmed to be closely associated with tumor occurrence, metastasis, invasion, recurrence, etc. Overexpression of CD133 often indicates poor prognosis for patients, making it significant for tumor treatment.

Macrophages, as important innate immune cells, mainly exert functions of “phagocytosis,” digestion, and antigen presentation. In the tumor microenvironment, macrophages polarize to the tumor-promoting M2 phenotype, which instead produces negative effects such as promoting tumor growth, invasion, metastasis, and angiogenesis, and participating in the formation of an immunosuppressive microenvironment. Chimeric Antigen Receptor-Macrophage (CAR-MΦ) therapy is another promising CAR technology for tumor immunotherapy following CAR-T cell therapy. Viral vectors, due to their high transfection and expression efficiency, are currently one of the effective tools for achieving high-efficiency expression of exogenous genes. However, due to limitations such as immunogenicity, lack of targeting, and DNA insertion length of viral vectors, they have gradually been replaced by various non-viral vectors, such as cationic liposomes, as the mainstream gene delivery carriers. Research has shown that CAR-MΦ can promote the re-education of tumor-associated macrophages, actively transforming from the M2 phenotype to the M1 phenotype, enhancing targeting ability toward tumor stem cells, reshaping the tumor microenvironment, and restoring the functions of “phagocytosis,” digestion, and antigen presentation.

### SUMMARY

The present invention provides an application of Chimeric Antigen Receptor-Macrophage (CAR-macrophage) in the field of glioma immunotherapy. By constructing a nano-carrier to achieve targeted delivery of the chimeric antigen receptor plasmid to macrophages, CD133-CAR-MΦ is constructed, enhancing the specific phagocytosis of macrophages against CD133 highly expressed brain tumor stem cells and polarization from M2 phenotype to M1 phenotype, thereby reshaping the tumor suppressive microenvironment and achieving specific killing of glioma stem cells, effectively treating glioma.

Based on the above results, the present invention provides the following technical solutions:
in a first aspect, the present invention provides a chimeric antigen receptor, comprising an extracellular domain, a transmembrane region, and an intracellular signal transduction domain; wherein the extracellular domain comprises a leader signal peptide segment (Leader), an antigen recognition domain (scFv), and a hinge region (Hinge); wherein the leader signal peptide segment is selected from CD8α Leader, and the antigen recognition domain (scFv) is derived from a monoclonal antibody against the tumor stem cell-specific marker CD133.

Preferably, the antigen recognition domain is derived from CD133 monoclonal antibody AC133 or clone 7, conferring specific recognition function to CAR cells and significantly enhancing affinity for specific antigens.

Preferably, the hinge region sequence is derived from one or more of IgG, CD8α, or CD28; further preferably, the hinge region is selected from CD8α.

Preferably, the transmembrane region is derived from one or more of CD4, CD8α, CD28, or CD3ζ; further preferably, the transmembrane structure is selected from CD8α.

Preferably, the intracellular domain is a signal transduction domain derived from one or more of FcεRIγ or CD3ζ; further preferably, the signal transduction domain is CD3ζ.

In a preferred embodiment of the above technical solutions, the chimeric antigen receptor's sequence from extracellular to intracellular segment sequentially includes CD8 leader signal peptide, anti-CD133 single-chain variable fragment, CD8α hinge region, CD8α transmembrane region, CD3ζ signal transduction domain, myc-tag marker gene, P2A, EF1α, and EGFP; specifically, a nucleic acid sequence encoding the chimeric antigen receptor is as shown in SEQ ID NO:1.

In a second aspect, the present invention provides an immune cell modified with the chimeric antigen receptor of the first aspect, wherein the immune cell includes but is not limited to one of T cells, NK cells, or macrophages.

In a preferred embodiment of the present invention, the immune cell is a macrophage, achieving induction of M1 phenotype of macrophages in the tumor microenvironment, thereby activating tumor immunity.

The immune cell modified with the chimeric antigen receptor achieves expression of the chimeric antigen receptor through a gene expression vector; in a validated embodiment of the present invention, the expression vector is a PiggyBac transposon, with CD68 as a promoter, containing a replication origin site, 3′ITR, 5′ITR, a polynucleotide sequence encoding the chimeric antigen receptor of the first aspect, and optionally, a selectable marker.

The above chimeric antigen receptor-modified immune cell applied to tumor immunotherapy can achieve in vivo editing of macrophages through nano-carrier delivery to obtain the corresponding chimeric antigen receptor-macrophages, wherein the nano-carrier is one or more of nano-micelles, cationic liposomes, or poly(beta-amino ester) polymers (polymer PBAE); in a validated embodiment of the present invention, the nano-carrier is a nano-micelle formed by self-assembly of amphiphilic polymers.

In a third aspect, the present invention provides an amphiphilic polymer, comprising a hydrophilic domain and a hydrophobic domain, wherein the hydrophilic domain includes a cationic sequence peptide and a nuclear localization peptide (NLS), and the hydrophobic domain is palmitic acid (PA).

The amphiphilic polymer of the above third aspect (hereinafter referred to as (PA)2 peptide) can form nano-micelles with the plasmid expressing the chimeric antigen receptor through self-assembly in solution, effectively loading the gene expression vector, enabling efficient delivery to target sites and lysosomal escape.

Preferably, the nuclear localization peptide has a sequence KKKPRVK (SEQ ID NO:2), and a specific sequence of the cationic sequence peptide is GRKKRRQRRR (SEQ ID NO:3).

In a specific embodiment of the above preferred technical solution, a structure of the amphiphilic polymer is: (PA)2-KGRKKRRQRRRKKKPRVK (SEQ ID NO:4), i.e., two molecules of palmitic acid connected a nuclear localization peptide through a cationic sequence.

In a fourth aspect, the present invention provides a nano-carrier for immune engineered cells, using the amphiphilic polymer of the third aspect as a nano-micelle carrier to load an expression vector containing the encoding sequence of the chimeric antigen receptor of the first aspect.

Preferably, a construction method of the nano-carrier is as follows: mixing a certain proportion of the amphiphilic polymer with the above expression vector in solution to form nano-micelles through amphiphilic self-assembly.

Further preferably, the specific construction method is as follows: dissolving the amphiphilic polymer in DMSO, adding an aqueous solution treated with diethyl pyrocarbonate (DEPC) and a solution of the above expression vector, and vortexing to obtain the above nano-micelles.

Preferably, the expression vector also has modifications with targeting groups; further preferably, the targeting groups are macrophage-specific affinity targeting group, including but not limited to mannose, dextran, etc.

In a specific embodiment of the present invention, the targeting group is a targeting group for the macrophage-specific target CD206, namely, citraconic anhydride-modified dextran-modified nano-micelles, to enhance specific targeting ability towards macrophages. In the above embodiment, the preparation method of the nano-carrier targeting macrophages is as follows: adding citraconic anhydride-modified dextran to a solution of the above nano-micelle, wherein an N/P ratio of dextran to the expression vector in the nano-micelles is 8˜12:1; further preferably is 9:1, 10:1, or 11:1.

In a fifth aspect, the present invention provides a use of the chimeric antigen receptor of the first aspect, the chimeric antigen receptor-modified immune cells of the second aspect, or the nano-carrier for immune engineered cells of the fourth aspect in the preparation of an anti-tumor drug.

The anti-tumor drug of the above fifth aspect includes but is not limited to drugs used for preventing, treating, or improving skin cancer, lung cancer, esophageal cancer, cervical cancer, uterine cancer, pancreatic cancer, breast cancer, renal cancer, ureteral cancer, bladder cancer, liver cancer, and glioma; further preferably, the anti-tumor drug is an anti-glioma drug.

Beneficial effects of one or more of the above technical solutions:
the present invention provides a method for in vivo and in vitro editing of Chimeric Antigen Receptor-Macrophage (CAR-MΦ). By constructing positively charged amphiphilic polymer (PA)2-KGRKKRRQRRR-NLS ((PA)2 peptide) self-assembled into nano-micelles loaded with chimeric antigen receptor plasmids, gene expression vectors can be effectively carried, achieving effective target site delivery and lysosomal escape. The above nano-drug delivery carrier effectively realizes the CAR editing and phenotype repolarization process of macrophages in vivo and in vitro. Applied to glioma treatment, it can achieve CAR editing and re-education of intratumoral macrophages, targeting tumor stem cells, reshaping the tumor suppressive microenvironment, and achieving specific killing of glioma stem cells, effectively treating glioma.

### BRIEF DESCRIPTION OF THE DRAWINGS

The accompanying drawings forming part of this specification are used to provide a further understanding of the present invention. The illustrative embodiments and their descriptions are used to explain the present invention and do not constitute improper limitations of the present invention.

FIG. 1 is a schematic diagram of the CD133-CAR gene expression vector constructed with PiggyBac transposon (PiggyBac-CD68 promoter-CD133-CAR) in Example 1.

FIG. 2 is a schematic diagram of the chimeric antigen receptor structure in Example 1.

FIG. 3 is a schematic diagram of the preparation of Nano-porter containing CAR plasmid in Example 1.

FIG. 4 shows confocal images of the subcellular location after co-incubation of macrophages with free pCAR or NP-CAR in Example 1.

FIG. 5 shows flow cytometric images of EGFP-positive BMDM cells treated with free pCAR or NP-CAR in Example 1.

FIG. 6 shows the phagocytosis of glioma cells by macrophages treated with free CAR plasmid, NP, or NP-CAR in Example 1.

FIG. 7 shows bioluminescence imaging images of treatment regimens using free CAR plasmid or NP-CAR respectively in Example 1.

FIG. 8 shows mouse survival examination under treatment regimens with free CAR plasmid or NP-CAR respectively in Example 1.

### DESCRIPTION OF THE PREFERRED EMBODIMENTS

It should be pointed out that the following detailed descriptions are all exemplary and intended to provide further explanation of the present invention. Unless otherwise specified, all technical and scientific terms used herein have the same meaning as commonly understood by one of ordinary skill in the art to which this invention belongs.

It should be noted that the terminology used here is for the purpose of describing particular embodiments only and is not intended to be limiting of the exemplary embodiments of the invention. As used herein, unless the context clearly indicates otherwise, singular forms are also intended to include plural forms, and it should also be understood that when the terms “comprising” and/or “including” are used in this specification, they specify the presence of features, steps, operations, devices, components, and/or combinations thereof.

To enable those skilled in the art to better understand the technical solutions of the present invention, the technical solutions of the present invention will be described in detail in conjunction with specific examples.

### Example 1

### 1. Construction of PiggyBac Vector Containing CD133-CAR Nucleic Acid Sequence

The CAR plasmid used in this example was a piggyBac transposon gene expression vector, including CD8α hinge region, CD8α transmembrane domain, and CD3ζ intracellular origin domain. The DNA sequence of the single-chain variable fragment (scFv) targeting CD133 antigen was derived from AC133 or clone 7. EGFP was fused with the EF1α promoter and separated by a P2A sequence to construct a CAR plasmid with a reporter protein.

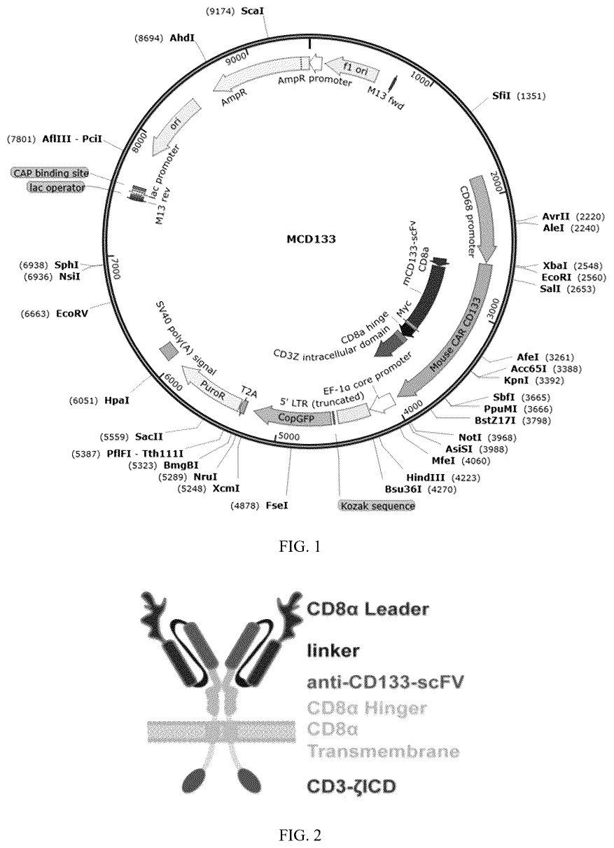

The plasmid map constructed in this example is shown in FIG. 1.

The chimeric antigen receptor structure constructed in this example is shown in FIG. 2, wherein the nucleic acid sequence encoding the chimeric antigen receptor is as shown in SEQ ID NO:1.

### 2. Preparation of Nano-Formulation

Nuclear localization sequence (NLS) peptide as the hydrophilic part and palmitic acid (PA) as the hydrophobic domain were used to construct a positively charged amphiphilic polymer (PA)2-KGRKKRRQRRR-NLS. The amphiphilic polymer could self-assemble into uniform nano-micelles with a critical micelle concentration (CMC) of 35.5 mg/L in aqueous solution. Negatively charged CAR plasmids were loaded into nano-micelles through electrostatic interaction. 2 mg of amphiphilic polymer was dissolved in 10 μL DMSO. The solution was diluted with 1 mL of aqueous solution treated with diethyl pyrocarbonate (1 mL of DEPC was added to 1 L of triple-distilled water, shaken, left at room temperature for several hours, then autoclaved to decompose DEPC into CO₂ and ethanol) and 35 μL of CAR plasmid aqueous solution. The mixture was vortexed (at a speed of 1000 rpm/min for 20 seconds) to obtain nano-micelles containing CAR plasmid. Then citraconic anhydride-modified dextran, which has targeting action for the macrophage-specific target CD206, was added to the nano-micelle solution to make the N/P ratio of citraconic anhydride-modified dextran to plasmid 10:1, and stirred at room temperature for 30 min to form Nano-porter (NP) containing CAR plasmid.

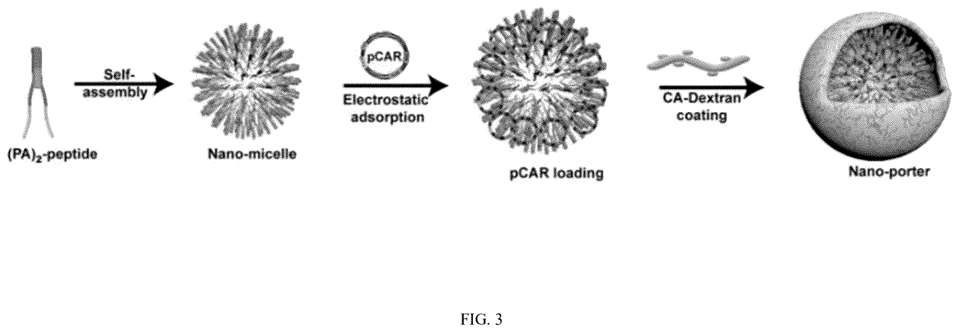

The schematic diagram of preparing Nano-porter containing CAR plasmid is shown in FIG. 3.

### 3. Uptake and Transfection of Nano-Porter by Macrophages

Cultured macrophages were prepared, and the qualitative and cellular uptake of NP-CAR was quantitatively assessed. Cells were first incubated with free CAR plasmid or NP-CAR at a plasmid dose of 5 μg/mL. After culture, for CLSM observation, lysosomes and cell nuclei were stained with Lysotracker and DAPI respectively, then analyzed by confocal laser scanning microscopy.

FIG. 4 shows confocal images of the subcellular location after co-incubation of macrophages with free CAR plasmid or NP-CAR. The results showed that the nano-formulation could effectively deliver genes to macrophages. With increasing incubation time, the plasmids were widely distributed in the cytoplasm. At the same time, only a small amount of plasmid was contained in endosomes/lysosomes within cells at 8 hours, indicating that the nano-formulation has lysosomal escape function.

For in vitro gene transfection experiments, BMDM cells were incubated with saline, free CAR plasmid, or NP-CAR respectively. After 48 h incubation, the percentage of EGFP-positive cells was detected by flow cytometry.

FIG. 5 shows the percentage of EGFP-positive BMDM cells treated with free CAR plasmid or NP-CAR. The results showed that the EGFR positive expression rate in the free CAR plasmid group was only 0.97%, while the EGFP positive expression rate in cells treated with NP-CAR was as high as 35.3%, improving the transfection effect by thirty-six fold.

### 4. Phagocytosis of Tumor Cells by Macrophages

BMDM cells pretreated with saline, free CAR plasmid, or NP-CAR were co-cultured with GL261 cells respectively. After 4 h of co-culture, cells were collected, stained with anti-CD11b, and then analyzed by flow cytometry.

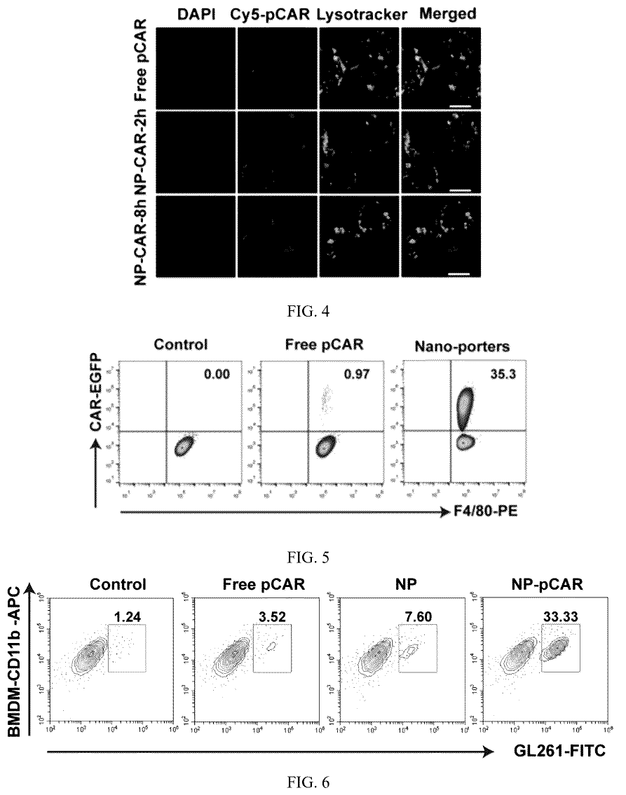

FIG. 6 shows the phagocytosis of glioma cells by macrophages treated with free CAR plasmid, NP, or NP-CAR, with the numbers in the figure representing the phagocytosis ratio of macrophages. The results showed that the phagocytosis ratio of tumor cells by macrophages treated with NP-CAR reached 33.33%, higher than the phagocytosis ratio of macrophages treated with free CAR plasmid or NP.

### 5. Establishment and Treatment of Mouse Brain Tumor Model

Using inhalation of 1%-5% isoflurane mixed with oxygen to anesthetize mice, an intracranial GBM mouse model was established by stereotaxically inoculating Luc+GL261 cells (150,000 cells in 7 μL PBS per mouse) into the brain. To determine the in vivo transfection efficiency, GL261-carrying mice were randomly divided into 3 groups, and different treatment regimen drugs were injected intratumorally 12 days after inoculation, with 3 mice used for bioluminescence imaging experimental studies and 6 mice for survival observation.

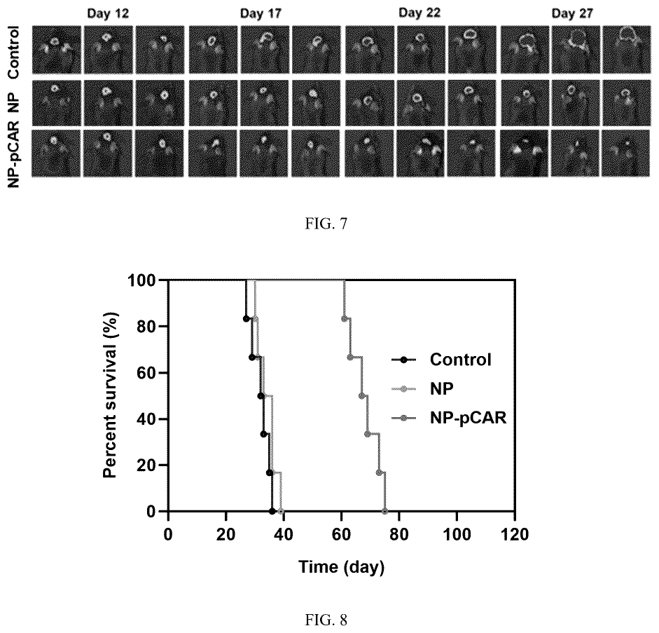

FIG. 7 shows bioluminescence imaging images of different treatment regimens. The results showed that the fluorescence intensity of tumor tissue in mice in the NP-CAR treatment group was lower than in other formulation groups, indicating that it could effectively inhibit tumor growth.

FIG. 8 shows mouse survival examination under different treatment regimens. The results showed that NP-CAR nano-formulation could effectively prolong survival, with a median survival of 68 days.

The above description is only the preferred embodiment of the present invention and is not used to limit the present invention. For those skilled in the art, the present invention can have various changes and variations. Any modifications, equivalent replacements, improvements, etc., made within the spirit and principles of the present invention should be included within the protection scope of the present invention.

1. A chimeric antigen receptor, comprising an extracellular domain, a transmembrane region, and an intracellular signal transduction domain, wherein the extracellular domain comprises a leader signal peptide segment, an antigen recognition domain, and a hinge region, wherein the leader signal peptide segment is selected from CD8α Leader, and the antigen recognition domain is derived from a monoclonal antibody against the tumor stem cell-specific marker CD133.

2. The chimeric antigen receptor according to claim 1, wherein the chimeric antigen receptor satisfies at least one condition selected from the group consisting of:
the antigen recognition domain is derived from CD133 monoclonal antibody AC133 or clone 7;
the hinge region sequence is derived from one or more of IgG, CD8α, or CD28;
the transmembrane region is derived from one or more of CD4, CD8α, CD28, or CD3ζ; and
the intracellular domain is a signal transduction domain derived from one or more of FcεRIγ or CD3ζ.

3. The chimeric antigen receptor according to claim 1, wherein the chimeric antigen receptor's sequence from extracellular to intracellular segment sequentially includes CD8 leader signal peptide, anti-CD133 single-chain variable fragment, CD8α hinge region, CD8α transmembrane region, CD3ζ signal transduction domain, myc-tag marker gene, P2A, EF1α, and EGFP.

4. An immune cell modified with the chimeric antigen receptor according to claim comprises at least 1, wherein the immune cell comprises at least one of T cells, NK cells, or macrophages.

5. The immune cell modified with the chimeric antigen receptor according to claim 4, wherein the immune cell is the macrophage;
wherein the immune cell modified with the chimeric antigen receptor achieves expression of the chimeric antigen receptor through a gene expression vector; wherein the expression vector is a PiggyBac transposon, with CD68 as a promoter, containing a replication origin site, 3′ITR, 5′ITR, a polynucleotide sequence encoding a chimeric antigen receptor comprising an extracellular domain, a transmembrane region, and an intracellular signal transduction domain, wherein the extracellular domain comprises a leader signal peptide segment, an antigen recognition domain, and a hinge region, wherein the leader signal peptide segment is selected from CD8α Leader, and the antigen recognition domain is derived from a monoclonal antibody against the tumor stem cell-specific marker CD133, and optionally, a selectable marker;
wherein the immune cell modified with the chimeric antigen receptor achieves in vivo editing of macrophages through nano-carrier delivery, wherein the nano-carrier is one or more of nano-micelles, cationic liposomes, or polymer PBAE.

6. An amphiphilic polymer, comprising a hydrophilic domain and a hydrophobic domain, wherein the hydrophilic domain comprises a cationic sequence peptide and a nuclear localization peptide, and the hydrophobic domain is palmitic acid.

7. A nano-carrier for immune engineered cells, wherein the nano-carrier uses the amphiphilic polymer according to claim 6 as a nano-micelle carrier to load an expression vector containing the encoding sequence of a chimeric antigen receptor comprising an extracellular domain, a transmembrane region, and an intracellular signal transduction domain, wherein the extracellular domain comprises a leader signal peptide segment, an antigen recognition domain, and a hinge region, wherein the leader signal peptide segment is selected from CD8α Leader, and the antigen recognition domain is derived from a monoclonal antibody against the tumor stem cell-specific marker CD133.

8. The nano-carrier for immune engineered cells according to claim 7, wherein the expression vector also has modifications with targeting groups.

9. A method for treating cancer in a patient in need thereof, comprising:
administering to the patient an effective amount of a therapeutic agent selected from the group consisting of:
(a) the chimeric antigen receptor according to claim 1,
(b) an immune cell modified with the chimeric antigen receptor, wherein the immune cell comprises at least one cell type selected from the group consisting of T cells, NK cells, and macrophages, and
(c) a nano-carrier for immune engineered cells, wherein the nano-carrier uses an amphiphilic polymer as a nano-micelle carrier to load an expression vector containing the encoding sequence of the chimeric antigen receptor, wherein the amphiphilic polymer comprises a hydrophilic domain and a hydrophobic domain, and the hydrophilic domain comprises a cationic sequence peptide and a nuclear localization peptide, and the hydrophobic domain is palmitic acid.

10. The method according to claim 9, wherein the cancer is selected from the group consisting of skin cancer, lung cancer, esophageal cancer, cervical cancer, uterine cancer, pancreatic cancer, breast cancer, renal cancer, ureteral cancer, bladder cancer, liver cancer, and glioma.

11. The chimeric antigen receptor according to claim 2, wherein the chimeric antigen receptor satisfies at least one condition selected from the group consisting of:
the hinge region sequence is derived from CD8α;
the transmembrane region is derived from CD8α, and
the intracellular domain is a signal transduction domain derived from CD3ζ.

12. The immune cell modified with the chimeric antigen receptor according to claim 5, wherein the nano-carrier is a nano-micelle formed by self-assembly of amphiphilic polymers.

13. The amphiphilic polymer according to claim 6, wherein the nuclear localization peptide has a sequence KKKPRVK; wherein a specific sequence of the cationic sequence peptide is GRKKRRQRRR.

14. The nano-carrier for immune engineered cells according to claim 7, wherein the nuclear localization peptide has a sequence KKKPRVK; wherein a specific sequence of the cationic sequence peptide is GRKKRRQRRR.

15. The nano-carrier for immune engineered cells according to claim 7, wherein a method for constructing of the nano-carrier comprises mixing a certain proportion of the amphiphilic polymer with the expression vector in solution to form nano-micelles through amphiphilic self-assembly.

16. The nano-carrier for immune engineered cells according to claim 7, wherein at least one of the following is satisfied:
the antigen recognition domain is derived from CD133 monoclonal antibody AC133 or clone 7;
the hinge region sequence is derived from one or more of IgG, CD8α, or CD28;
the transmembrane region is derived from one or more of CD4, CD8α, CD28, or CD3ζ; and
the intracellular domain is a signal transduction domain derived from one or more of FcεRIγ or CD3ζ.

17. The nano-carrier for immune engineered cells according to claim 7, wherein the chimeric antigen receptor's sequence from extracellular to intracellular segment sequentially includes CD8 leader signal peptide, anti-CD133 single-chain variable fragment, CD8α hinge region, CD8α transmembrane region, CD3ζ signal transduction domain, myc-tag marker gene, P2A, EF1α, and EGFP.

18. The nano-carrier for immune engineered cells according to claim 8, wherein the targeting groups are macrophage-specific affinity targeting groups selected from mannose and dextran.

19. The nano-carrier for immune engineered cells according to claim 8, wherein the targeting group is citraconic anhydride-modified dextran.

20. The method according to claim 10, wherein the cancer is glioma.
